(19) United States
(12) Patent Application Publication
(10) Pub. No.: US 2024/0058752 A1
(43) Pub. Date: Feb. 22, 2024

(54) CONTINUOUS CARBON DIOXIDE CONVERSION PROCESS, AND SYSTEM THEREFOR

(71) Applicant: Korea University Research and Business Founess Foundation, Seoul (KR)

(72) Inventors: Jungbae KIM, Seoul (KR); Joongbok OK, Seoul (KR); Seungkeun KIM, Seoul (KR); Yunjae kim, Seoul (KR); Han Sol KIM, Yongin-si (KR)

(73) Assignee: Korea University Research and Business Founess Foundation, Seoul (KR)

(21) Appl. No.: 18/270,093

(22) PCT Filed: Dec. 29, 2020
(86) PCT No.: PCT/KR2020/019314

§ 371 Date: Jun. 28, 2023

(30) Foreign Application Priority Data
Dec. 29, 2020 (KR) 10-2020-0186040

(51) Int. Cl. B01D 53/84; B01D 53/62; B01D 53/78; B01D 53/96; C01F 11/18; C12P 3/00

(52) U.S. Cl. CPC B01D 53/84; B01D 53/62; B01D 53/78; B01D 53/96; C01F 11/18; C12P 3/00; B01D 2258/0283; B01D 2257/504; B01D 2251/95

(57) ABSTRACT

The present invention relates to a carbon dioxide conversion process and, more particularly, to a continuous carbon dioxide conversion process and a system therefor.

### TECHNICAL FIELD

The present disclosure relates to a carbon dioxide conversion process, and more particularly, to a continuous carbon dioxide conversion process and a system therefor.

### BACKGROUND ART

Global warming is one environmental problem that continuously comes up every year. As one way to overcome global warming, a technology of collecting and converting carbon dioxide, which is a greenhouse gas, has been proposed. However, a typical carbon dioxide conversion technology has problems in that, due to requiring a large amount of energy when converting a greenhouse gas, greenhouse gas emission is rather induced, and because a production cycle of a material obtained through conversion is short, carbon dioxide is emitted again. Accordingly, a successful carbon dioxide conversion system should be able to reduce the total amount of emitted carbon dioxide in the entire process, and a new process utilizing carbon dioxide should be a system having a technology that uses less energy and resources compared to the conventional alternative process.

Generally, energy used in the process of converting carbon dioxide is secondary energy introduced from outside the process and, in most cases, is energy modified or processed from nature or initial energy. Securing business competitiveness through cost reduction is one issue that has been mentioned in recent years in various processes including the chemical industry, and due to the occurrence of various social problems relating to resource depletion and environmental pollution, various studies are being conducted to optimize process operations using less energy in the process. In particular, among operation costs for a chemical process, energy costs account for the next largest portion after raw material costs, and thus there is an urgent need to reduce energy costs.

Meanwhile, even when the efficiency of a carbon dioxide conversion reaction is high, in order to separate a primary product generated through the conversion reaction or facilitate the separation of the primary product, a process of separating a converted secondary product is essential. However, in general, it is not easy to simultaneously perform the conversion process and the primary product separation process or the converted secondary product separation process, and thus there is a problem that the carbon dioxide conversion reaction should be stopped to perform the separation process, and there is a problem that energy and time are consumed for a separately-performed separation process.

Accordingly, there is an urgent need for research on carbon dioxide conversion technology that can maximize conversion efficiency while reducing energy and resources, which are consumed in large amounts in conversion processes incorporated in the conventional carbon dioxide conversion technologies, and reducing the time and cost required for the process of separating converted products.

### DISCLOSURE

### Technical Problem

The present disclosure is directed to providing a continuous carbon dioxide conversion process and a system therefor capable of reducing the amount of water and energy required for a carbon dioxide conversion process and converting carbon dioxide with excellent efficiency.

The present disclosure is also directed to providing a continuous carbon dioxide conversion process and a system therefor capable of simultaneously performing a process of converting carbon dioxide into a primary product and a process of separating the primary product without requiring separate resources or time for the separation process, thus being able to continuously perform a carbon dioxide conversion process with excellent efficiency and reduced costs.

### Technical Solution

The present disclosure provides a continuous carbon dioxide conversion process including a first process in which carbon dioxide introduced into a first reaction portion is converted into bicarbonate ions through a conversion reaction portion including a liquid, which includes water, and carbonic anhydrase positioned in the vicinity of the surface of the liquid, a new liquid is supplied into the first reaction portion, and a liquid exceeding a predetermined liquid level in the first reaction portion due to the supplied new liquid overflows and is supplied to a second reaction portion, a second process in which the liquid containing bicarbonate ions, which is introduced into the second reaction portion through the first process, is regenerated into a liquid from which the bicarbonate ions are removed through a liquid regeneration portion in the second reaction portion, and a third process in which the regenerated liquid recovered through the second process is directly or indirectly resupplied to the first reaction portion.

According to one embodiment of the present disclosure, the new liquid may be supplied from a liquid storage to the first reaction portion, and in the third process, the regenerated liquid may be indirectly resupplied by being supplied to the first reaction portion after being supplied to the liquid storage.

Also, in order to control both a flow rate of the new liquid supplied from the liquid storage to the first reaction portion and a flow rate of the liquid supplied from the first reaction portion to the second reaction portion through a single first pump, the liquid overflowing from the first reaction portion and being supplied to the second reaction portion may be supplied by natural drainage, and in the third process, the regenerated liquid may be supplied to the liquid storage through natural drainage.

Also, the new liquid may be supplied to the first reaction portion via an intermediate liquid storage disposed between the liquid storage and the first reaction portion, and in order to control both a flow rate of the new liquid supplied from the liquid storage to the first reaction portion and a flow rate of the liquid supplied from the first reaction portion to the second reaction portion through a single first pump, the new liquid supplied from the intermediate liquid storage to the first reaction portion and the liquid supplied from the first reaction portion to the second reaction portion may each be supplied by natural drainage, and in the third process, the regenerated liquid may be supplied to the liquid storage through natural drainage.

Also, the carbon dioxide introduced into the first reaction portion may be carbon dioxide contained in a flue gas or carbon dioxide separated and refined from a flue gas.

Also, the carbon dioxide introduced into the first reaction portion may have a gas flow in which a residual amount unconverted after being introduced from a portion above the surface of the liquid flows out above the surface of the liquid.

Also, in order to position the carbonic anhydrase in the vicinity of the liquid surface without being affected by a change in liquid level and prevent loss of the carbonic anhydrase at the time of discharge of the overflowing liquid, the carbonic anhydrase may be provided to be fixed to a floating body, and the floating body may be provided to not exit the first reaction portion.

Also, the vicinity of the liquid surface may be a liquid region from the surface to a point where a depth is 10 cm.

Also, in order to increase a rate at which carbon dioxide is converted into bicarbonate ions in the first reaction portion, the flow rate of the new liquid supplied into the first reaction portion may be determined as a flow rate that exceeds a flow rate at the time the value of R according to Equation 1 below satisfies 1.

(Amount of calcium carbonate produced in carbon dioxide conversion system A (g))/(Amount of calcium carbonate produced in carbon dioxide conversion system B (g))=R [Equation 1]

Here, the carbon dioxide conversion system A is a carbon dioxide conversion system in which the conversion reaction portion including the carbonic anhydrase is disposed in the first reaction portion, the carbon dioxide conversion system B is a carbon dioxide conversion system identical to the carbon dioxide conversion system A except for the absence of the carbonic anhydrase in the first reaction portion, and the amount of calcium carbonate produced in the two carbon dioxide conversion systems refers to the amount of calcium carbonate produced in the second reaction portion a predetermined time after the start of supply of carbon dioxide and the new liquid with a predetermined flow rate to the first reaction portion in a state in which the first reaction portion is filled with a liquid up to the highest possible liquid level at which the liquid does not overflow, and calcium ions are provided in the liquid regeneration portion of the second reaction portion.

Also, in order to disperse the bicarbonate ions obtained by conversion in the first process, the liquid in the first reaction portion may be stirred in the first process.

Also, the liquid regeneration portion may include calcium ions, and by the bicarbonate ions introduced into the liquid regeneration portion being ultimately converted into calcium carbonate through the calcium ions, the bicarbonate ions may be removed.

Also, the second process may be run in a batch circulation manner in which a plurality of second reaction portions each having the liquid regeneration portion containing calcium ions are sequentially added to the second process, and the second reaction portion that has completed the second process is added to the second process again.

Also, the second reaction portion that has completed the second process may further perform a regenerated liquid discharge process in which the regenerated liquid, excluding a calcium carbonate precipitate, in the second reaction portion is separated and discharged, a precipitate discharge process in which the calcium carbonate precipitate is discharged from the second reaction portion from which the liquid has been discharged, and a selective regenerated liquid processing process in which, as a result of measuring a concentration of calcium ions in the regenerated liquid, the regenerated liquid is supplied to another second reaction portion, which is in a state in which both the liquid and the precipitate have been discharged, and then added to the second process again in a case in which the calcium ions are contained at a predetermined concentration or higher, or the regenerated liquid is added to the third process in a case in which the calcium ions are contained at a concentration lower than the predetermined concentration.

The present disclosure provides a continuous carbon dioxide conversion system including a first reaction portion including a first chamber having an empty space therein, a conversion reaction portion including a liquid, which includes water, with which the empty space is filled up to a predetermined liquid level, and carbonic anhydrase positioned in the vicinity of the surface of the liquid, a first inlet disposed at one side of the first chamber to receive a new liquid, and a first outlet provided to allow a liquid exceeding the predetermined liquid level to overflow and be drained, a second reaction portion including a second chamber having an empty space therein, a second inlet through which the liquid drained after overflowing from the first reaction portion is introduced, a liquid regeneration portion configured to remove bicarbonate ions contained in the introduced liquid and regenerate the liquid, and a second outlet configured to discharge the regenerated liquid, and a pump configured to directly or indirectly supply the regenerated liquid, discharged through the second outlet, to the first reaction portion.

According to one embodiment of the present disclosure, the first reaction portion may further include a carbon dioxide inlet and a carbon dioxide outlet provided in the first chamber so that carbon dioxide has a gas flow in which a residual amount unconverted after being introduced from a portion above the surface of the liquid flows out above the surface of the liquid.

Also, a height from the ground to the first outlet may be formed higher than a height from the ground to the second inlet so that the liquid overflowing from the first reaction portion is naturally drained into the second reaction portion.

Also, the first reaction portion may further include a stirring portion to disperse bicarbonate ions, obtained by conversion, from the conversion reaction portion into the surrounding liquid.

Also, in the second reaction portion, the liquid regeneration portion may contain calcium ions, a calcium carbonate outlet configured to discharge a calcium carbonate precipitate, obtained by the bicarbonate ions in the introduced liquid being ultimately converted through a reaction with the calcium ions, may be further included, and the second outlet may be disposed at a higher point than a thickness of the calcium carbonate precipitate in order to facilitate separation and discharge of the remaining regenerated liquid excluding the calcium carbonate precipitate.

Also, the continuous carbon dioxide conversion system may further include a liquid storage which is a supply source of the new liquid supplied to the first reaction portion and a first pump disposed on a conduit connecting the liquid storage and the first reaction portion, and a second pump as a pump for delivering the regenerated liquid, discharged through the second outlet, to the liquid storage or a structural device designed to deliver the regenerated liquid to the liquid storage through natural drainage may be provided.

In addition, the second reaction portion may be provided as a plurality of second reaction portions, and a moving device configured to move the plurality of second reaction portions so that, after any one second reaction portion finishes receiving a liquid from the first reaction portion, another second reaction portion sequentially receives a liquid from the first reaction portion may be further provided.

### Advantageous Effects

A continuous carbon dioxide conversion process according to the present disclosure converts carbon dioxide gas using carbonic anhydrase and thus can minimize energy required for conversion. Also, by preventing a decrease in conversion process efficiency due to accumulation of a primary product generated by the carbon dioxide conversion process, carbon dioxide can be converted while continuously maintaining the efficiency at a certain level or higher. Further, the primary product obtained by conversion can be continuously separated in a reactor in which the conversion process is performed, without requiring a separate process, energy, and time, and the separated primary product can also be moved without requiring separate energy, and thus it is easy to separate and discharge the primary product generated in the carbon dioxide conversion process, and the required energy can be reduced. Also, since it is not necessary to stop the conversion process to separate the primary product or to charge water required in the conversion process after the separation of the primary product, it is possible to continuously convert carbon dioxide. Further, by regenerating and recirculating a large amount of water used in the carbon dioxide conversion process, the amount of consumed water resources can be significantly reduced, and in this way, relative to the amount of used water resources, the amount of produced primary product or secondary product, such as calcium carbonate, obtained by conversion of the primary product can be maximized, and thus there is an advantage in terms of economic feasibility and environmental friendliness.

### DESCRIPTION OF DRAWINGS

FIG. 1 is a schematic diagram of a continuous carbon dioxide conversion system according to one embodiment of the present disclosure.

FIG. 2 is a schematic diagram of the flow of carbon dioxide flowing into and out of a first reaction portion according to another embodiment of the present disclosure.

FIG. 3 is a schematic diagram of the position of carbonic anhydrase employed in the continuous carbon dioxide conversion system according to one embodiment of the present disclosure.

FIG. 4 is a view illustrating a liquid supplied into the first reaction portion and a liquid overflowing and being discharged to a second reaction portion in the continuous carbon dioxide conversion system according to FIG. 1.

FIG. 5 is a partial schematic diagram illustrating the supply of a liquid, drained after overflowing from the first reaction portion, to the second reaction portion according to another embodiment of the present disclosure.

FIG. 6 is a partial schematic diagram of a carbon dioxide conversion system in which a second process is run in a batch circulation manner according to another embodiment of the present disclosure.

FIGS. 7 and 8 are schematic diagrams of a carbon dioxide conversion system according to another embodiment of the present disclosure.

FIG. 9 is a graph showing the amount of calcium carbonate produced in the second reaction portion for each residence time of a liquid in the first reaction portion when running a carbon dioxide conversion system according to Preparation Example 1 and Preparation Example 2 of the present disclosure.

### BEST MODE OF THE INVENTION

Hereinafter, embodiments of the present disclosure will be described in detail to allow those of ordinary skill in the art to which the present disclosure pertains to easily carry out the present disclosure. The present disclosure may be implemented in various different forms and is not limited to the embodiments described herein.

A continuous carbon dioxide conversion process according to one embodiment of the present disclosure includes a first process in which carbon dioxide introduced into a first reaction portion is converted into bicarbonate ions through a conversion reaction portion including a liquid, which includes water, and carbonic anhydrase positioned in the vicinity of the surface of the liquid, a new liquid is continuously supplied into the first reaction portion, and a liquid exceeding a predetermined liquid level in the first reaction portion due to a liquid level rise caused by the supplied new liquid overflows and is supplied to a second reaction portion, a second process in which the liquid containing bicarbonate ions, which is introduced into the second reaction portion through the first process, is regenerated into a liquid from which the bicarbonate ions are removed through a liquid regeneration portion in the second reaction portion, and a third process in which the regenerated liquid recovered through the second process is directly or indirectly resupplied to the first reaction portion.

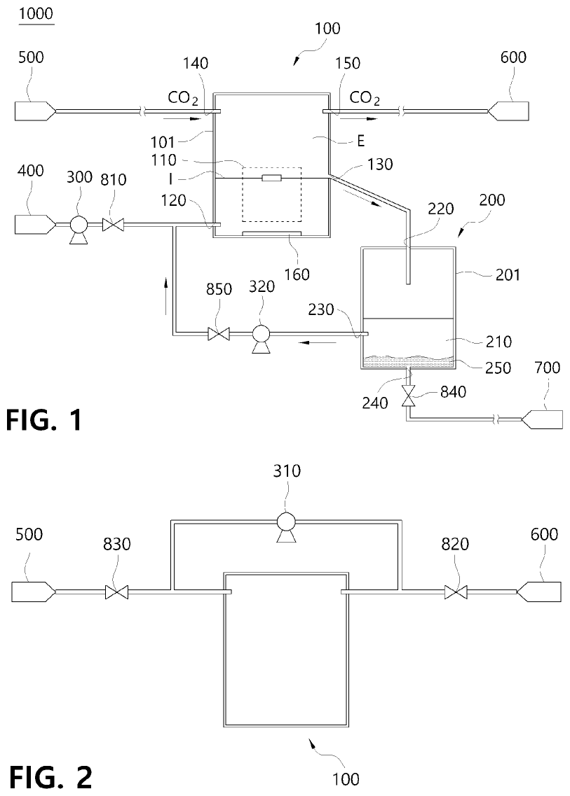

Referring to FIGS. 1 to 5, first, the first process, which is a process in which carbon dioxide introduced into the first reaction portion is converted into bicarbonate ions through a reaction and the bicarbonate ions obtained by conversion are discharged to the outside of the first reaction portion, is performed in a first reaction portion 100 in a carbon dioxide conversion system 1000.

In order to perform the first process, the first reaction portion 100 includes a first chamber 101 having an empty space E therein to accommodate a liquid 111, a first inlet 120 through which the liquid 111 is supplied from the outside into the first chamber 101, a first outlet 130 through which the liquid 111 accommodated in the first chamber 101 is drained to the outside, a carbon dioxide inlet 140 through which carbon dioxide is supplied from the outside, a carbon dioxide outlet 150 through which a residual amount of carbon dioxide that remains after conversion is discharged to the outside, and a conversion reaction portion 110 configured to convert carbon dioxide into bicarbonate ions.

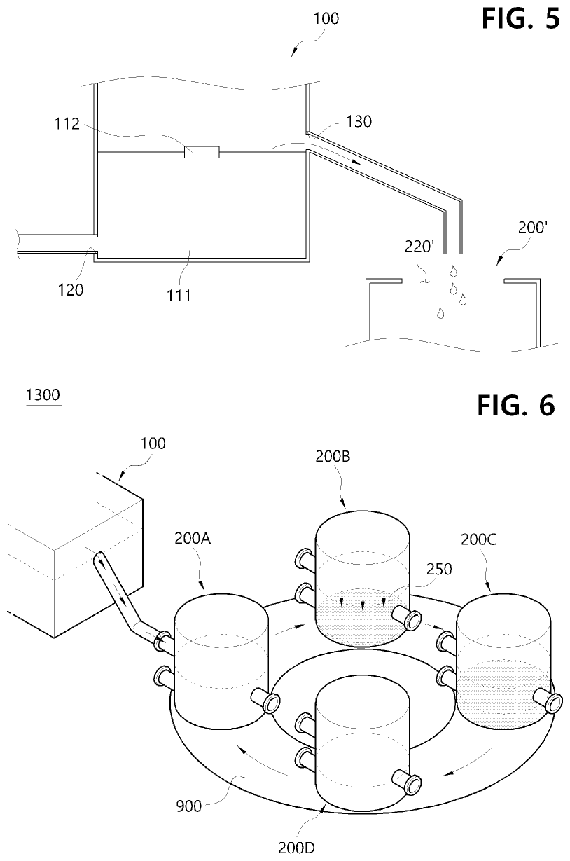

Specifically, carbon dioxide has a gas flow in which a residual amount of carbon dioxide, which is is unreacted after being introduced from a carbon dioxide supply source 500 into the first reaction portion 100, is discharged to the outside of the first reaction portion. Here, the carbon dioxide may be introduced into the first reaction portion 100 through the carbon dioxide inlet 140 formed at one side of the first reaction portion 100. Specifically, the carbon dioxide may have a first gas flow in which carbon dioxide is introduced from a portion above the liquid 111 and flows out above the surface of the liquid again (see FIG. 1). Alternatively, as in a first reaction portion 100′ of a carbon dioxide conversion system 1000′ illustrated in FIG. 5, carbon dioxide may be implemented to have a second gas flow in which carbon dioxide is introduced to pass through a liquid and then flows out above the surface of the liquid. However, carbon dioxide having the first gas flow may be desirable for maintaining a concentration of bicarbonate ions obtained by conversion in the vicinity of the conversion reaction portion 110 at a low level. In the case of the second gas flow, in a path along which carbon dioxide passes through the liquid and reaches the conversion reaction portion 110 in the vicinity of the surface of the liquid, some carbon dioxide may be hydrated and converted into bicarbonate ions, and it may be difficult to lower the concentration of bicarbonate ions in the vicinity of the conversion reaction portion 110 to a certain level or lower, and thus there is a concern that the efficiency of a conversion reaction using carbonic anhydrase 112 may decrease. Also, it may be difficult to separate and discharge bicarbonate ions, converted in liquid at greater depths instead of near the liquid surface, through natural drainage through an overflow.

Also, the carbon dioxide supply source 500 may be a storage tank in which separated and refined carbon dioxide is collected, a storage tank in which discharged flue gas is collected, a flue gas generation source, or the atmosphere, and accordingly, carbon dioxide introduced into the first reaction portion 100 may be separated and refined carbon dioxide, a flue gas, or atmospheric air.

Also, carbon dioxide introduced into the first reaction portion 100 may be converted into bicarbonate ions through the conversion reaction portion 110 on a gas flow path, a residual amount of carbon dioxide that is unconverted may be discharged to a carbon dioxide collection source 600 through the carbon dioxide outlet 150, and the carbon dioxide collection source 600 may be a storage tank or the atmosphere. According to another embodiment of the present disclosure, as illustrated in FIG. 2, carbon dioxide may have a closed type gas flow in which carbon dioxide discharged from the carbon dioxide supply source 500 is introduced into the first reaction portion 100 for a predetermined amount of time, and then carbon dioxide flow control valves 820 and 830 are closed to convert the entire amount of carbon dioxide, and carbon dioxide discharged from the first reaction portion 100 is reintroduced into the first reaction portion 100 using a gas pump 310.

Also, the conversion reaction portion 110 serves to convert carbon dioxide introduced into the first reaction portion 100 into bicarbonate ions. The conversion reaction portion 110 includes the liquid 111, which includes water, and the carbonic anhydrase 112.

In the conversion reaction portion 110, the carbonic anhydrase 112 is disposed to be positioned in the vicinity of the surface of the liquid, and in this way, it is advantageous for increasing the possibility of contact between carbon dioxide, flowing in a space E above the surface, and the liquid 111 and the carbonic anhydrase to maximize carbon dioxide conversion efficiency. Also, bicarbonate ions obtained by conversion may be contained at a high concentration around the carbonic anhydrase 112, and the concentration of bicarbonate ions may be high in the vicinity of the surface, but as will be described below, when the liquid 111 exceeds a predetermined liquid level a due to the supply of the new liquid, the liquid exceeding the liquid level a, that is, the liquid near the surface, may overflow and be drained, and, at this time, bicarbonate ions obtained by conversion may also be discharged together, and thus there is an advantage that it is easy to separate and discharge the bicarbonate ions, obtained by conversion, from the first reaction portion 100, and a separate bicarbonate ion separation process may be omitted. Also, because the bicarbonate ions obtained by conversion are discharged through an overflow from the first reaction portion 100, the concentration of bicarbonate ions around the carbonic anhydrase 112 may be lowered, and in this way, it is advantageous for minimizing or preventing a reduction in carbon dioxide conversion efficiency.

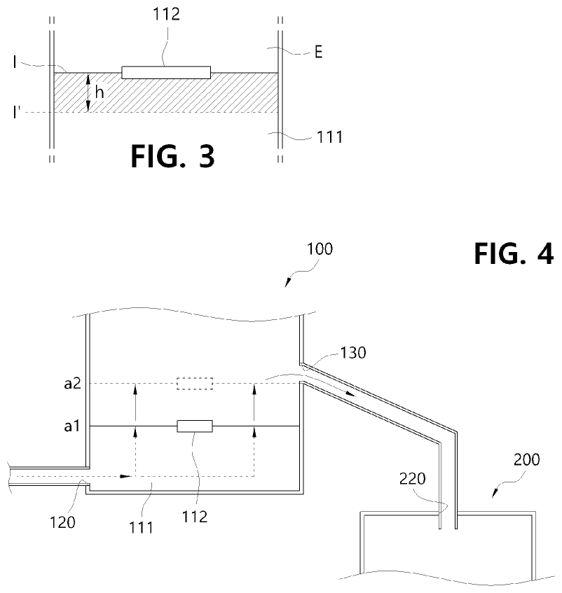

Here, the vicinity of the surface may be a liquid region from a surface I to a point I′ which is at a predetermined depth h from the surface I as illustrated in FIG. 3. For example, the vicinity of the surface may be a liquid region from the surface to a point at a depth of 2 cm from the surface in the case of a calm surface without waves and may be a liquid region from the surface to a point at a depth of 10 cm from the surface in the case in which waves are present. However, it should be noted that the depth from the surface may be changed according to whether the height of waves is high or low, and the depth from the surface may increase when the height of waves is high. In a case in which the carbonic anhydrase is positioned at a depth of more than 10 cm from the surface, there is a concern that carbon dioxide conversion efficiency may decrease. Also, bicarbonate ions obtained by conversion in water may be contained in a liquid overflowing due to a continuous liquid supply, which will be described below, and may be separated and discharged to a second reaction portion 200, but it may be difficult for bicarbonate ions obtained by conversion in liquid at a depth exceeding a predetermined depth to be contained in an overflowing liquid and discharged, and when even bicarbonate ions obtained by conversion in liquid at greater depths are discharged through an overflow, because a large amount of liquid containing bicarbonate ions at a low concentration needs to overflow, it is required to drain and resupply an excessive amount of liquid, which is undesirable. Also, in a case in which bicarbonate ions obtained by conversion in liquid at greater depths are not able to be discharged through an overflow, there is a problem that a separation process should be separately performed to separate such bicarbonate ions. Also, in a case in which the amount of bicarbonate ions failing to overflow increases, the efficiency of carbon dioxide conversion through the conversion reaction portion 110 decreases, and there is a concern that the bicarbonate ions obtained by conversion may rather be converted back to carbon dioxide.

The liquid 111 mediates a reaction of converting carbon dioxide into bicarbonate ions and/or serves as a reactant of the conversion reaction, and any liquid may be used without limitation as long as the bicarbonate ions obtained by conversion can be dissolved in the liquid without a problem. Accordingly, the liquid includes water and may further include a typical buffer solution, and as a nonlimiting example of the buffer solution, 2-amino-2-hydroxymethyl-1,3-propanediol may be used.

Also, generally, the carbonic anhydrase 112 may be an enzyme naturally present in a living body, such as an animal or a plant, and thus may be one selected from the group consisting of α-type, β-type, γ-type, δ-type and ε-type and/or carbonic anhydrase that mimics an enzyme that is present in vivo, carbonic anhydrase obtained by artificially recombining the enzyme, or a combination of these and carbonic anhydrase that is present in vivo. Since the carbonic anhydrase obtained by artificial recombination may be known, the amino sequence thereof is not particularly limited in the present disclosure.

Meanwhile, in order to be easily positioned in the vicinity of the surface (between the surface I and the point I′) described above, the carbonic anhydrase 112 may be provided in the conversion reaction portion 110 in a state in which the carbonic anhydrase 112 is fixed to a support body. The support body may be a floating body in order to correspond to a change in liquid level of the liquid 111 without separate energy and power consumption. However, the present disclosure is not limited thereto, and a support body that is not a floating body may have a separate floating body coupled thereto, or while a support body that is not a floating body is used, a separate position controller that allows the position of the support body to be artificially adjusted using power may be coupled to the support body.

Also, the support body may have a known shape such as a plate shape and may have a porous structure or a nonporous structure. Since the shape and structure of the support body may be changed in various ways according to the form and purpose of fixing the support body to carbonic anhydrase, the shape and structure of the support body are not limited by the present disclosure.

The carbonic anhydrase 112 may be directly or indirectly fixed to the above-described support body. Here, direct fixation refers to a case in which the carbonic anhydrase 112 is directly fixed to the support body physically or chemically without the interposition of another material. Here, examples of physical fixation may include adsorption of the carbonic anhydrase 112 or using a structural factor of the support body, for example, carrying the carbonic anhydrase 112 in a pore in the support body so that the carbonic anhydrase 112 is not able to exit the pore. Also, for example, chemical fixation may be an ionic bond or a covalent bond. Also, the indirect fixation refers to a case in which the carbonic anhydrase 112 is fixed using a third material such as a spacer, an adhesive, or a linker. For example, the indirect fixation may be fixation using a catechol group-based adhesive material such as polydopamine and polynorepinephrine or using a linker, e.g., a linker combination such as biotin-avidin, biotin-neutravidin, biotin-streptavidin, and digoxigenin-anti-digoxigenin. Also, the spacer may be beads, fibers, or the like, and a spacer to which the carbonic anhydrase 112 is fixed may be fixed again by the support body.

According to one embodiment of the present disclosure, the carbonic anhydrase 112 may be provided to be fixed to a floating structural body, and Korean Patent Application No. 10-2016-0080437 filed by the inventor of the present disclosure may be referenced for the floating structural body.

Meanwhile, the carbonic anhydrase 112 may be provided as a plurality of carbonic anhydrases 112, and at least some of the plurality of carbonic anhydrases 112 may be provided to be adsorbed to each other or may be provided to be bound to each other with a typical crosslinking material interposed therebetween. Specifically, according to one exemplary embodiment of the present disclosure, optimal conditions (temperature, pH, and the like) for a reaction of converting carbon dioxide into bicarbonate ions and optimal conditions for enzyme activity of the carbonic anhydrases 112 may be different, and in some cases, the enzyme activity of the carbonic anhydrases 112 may be difficult to maintain in a certain environment. Thus, the carbonic anhydrases 112 may be fixed in the form of an aggregate to a spacer such as fibers including a first functional group on a surface thereof, and specifically, an aggregate may be formed by at least some of the carbonic anhydrases being directly bound to the first functional group and the rest of the carbonic anhydrases being bound to any one or more of some of the carbonic anhydrases and the remaining carbonic anhydrases adjacent thereto.

Here, for the functional group provided on a surface of the spacer, any functional group may be used without limitations as long as the functional group can fix carbonic anhydrase. For example, the functional group may be any one or more selected from the group consisting of a carboxyl group, an amine group, an imine group, an epoxy group, a hydroxy group, an aldehyde group, a carbonyl group, an ester group, a methoxy group, an ethoxy group, a peroxy group, an ether group, an acetal group, a sulfide group, a phosphate group, and an iodide group and, preferably, may be any one or more of a carboxyl group and an amine group.

Here, production methods disclosed in Korean Unexamined Patent Application Publication Nos. 10-2011-0128128, 10-2011-0128134, and 10-2013-0127916 filed by the inventor of the present disclosure may be referenced for examples of an aggregate of carbonic anhydrases that is produced using a crosslinking agent.

Meanwhile, a reaction in which carbon dioxide is converted into bicarbonate ions in the liquid 111 including water is a reversible reaction, and because a reverse reaction in which bicarbonate ions are converted back to carbon dioxide is much easier to occur than a forward reaction in which carbon dioxide is converted into bicarbonate ions, in a case in which bicarbonate ions obtained by conversion around the conversion reaction portion 110 are not rapidly dispersed, separated, and/or discharged, the bicarbonate ions may be converted back to carbon dioxide, or the efficiency of the conversion reaction in the conversion reaction portion 110 may decrease. Accordingly, in the first process, it is important to rapidly disperse, separate, and/or discharge the converted carbon dioxide from the first reaction portion 100 to lower the concentration of bicarbonate ions contained near the liquid surface around the conversion reaction portion 110 or continue to maintain a state in which the concentration is low.

In the present disclosure, to this end, a new liquid is supplied into the first reaction portion 100, and when a liquid level rising due to the supplied new liquid exceeds a predetermined liquid level a₂, a liquid near the liquid surface overflows into the second reaction portion and is drained to the outside of the first reaction portion 100. In this way, there is an effect of separating bicarbonate ions, obtained by conversion near the liquid surface, from the first reaction portion 100 and releasing the bicarbonate ions to the outside, and there is an advantage that, by lowering the concentration of the bicarbonate ions around the conversion reaction portion 110, a reduction in efficiency of the reaction of converting carbon dioxide into bicarbonate ions can be minimized or prevented.

Specifically, referring to FIG. 4, when a liquid is supplied through the first inlet 120 of the first reaction portion 100 accommodating the liquid 111 up to a first liquid level a₁, with an effect of reducing the concentration of bicarbonate ions near the liquid surface, the liquid level may reach the predetermined liquid level a₂ which is at the same height as the first outlet 130, and even afterwards, a liquid exceeding the predetermined liquid level a₂ due to continuous supply of the liquid 111 may overflow and be drained to the outside of the first reaction portion 100, without separate energy consumption for drainage. Also, the first process of the first reaction portion 100 may be continuously run without pause when a new liquid is continuously supplied, the second process may also be continuously run without pause because the second reaction portion 200 can continuously receive the liquid containing bicarbonate ions from the first reaction portion 100, and the first process and the second process may be continuously performed. Meanwhile, the first inlet 120 may be disposed at one side of the first chamber 101 and may be disposed at a lower end of one side of the first chamber 101 as illustrated in FIG. 1, but the present disclosure is not limited thereto, and it should be noted that the first inlet 120 may also be provided above the surface of the liquid, specifically, above or at an upper surface of one side surface of the first chamber 101.

According to one exemplary embodiment of the present disclosure, by drainage through an overflow, the concentration of bicarbonate ions near the liquid surface may be lowered to ultimately increase a rate at which carbon dioxide is converted into bicarbonate ions, and in order to continuously maintain the increased conversion rate, a flow rate of the liquid supplied into the first reaction portion may exceed a flow rate at the time the value of R according to Equation 1 below is 1. When the liquid is supplied with a flow rate lower than the flow rate at the time the value of R according to Equation 1 below is 1, carbon dioxide conversion efficiency may decrease despite including carbonic anhydrase, and there is a concern that a separate bicarbonate ion separation process may have to be performed after stopping the conversion reaction in the conversion reaction portion 110 in order to lower the overall concentration of bicarbonate ions in the first reaction portion 100.

(Amount of calcium carbonate produced in carbon dioxide conversion system A (g))/(Amount of calcium carbonate produced in carbon dioxide conversion system B (g))=R [Equation 1]

Here, the carbon dioxide conversion system A is a carbon dioxide conversion system in which the conversion reaction portion including the carbonic anhydrase is disposed in the first reaction portion, the carbon dioxide conversion system B is a carbon dioxide conversion system identical to the carbon dioxide conversion system A except for the absence of the carbonic anhydrase in the first reaction portion, and the amount of calcium carbonate produced in the two carbon dioxide conversion systems refers to the amount of calcium carbonate produced in the second reaction portion a predetermined time after the start of supply of carbon dioxide and the new liquid with a predetermined flow rate to the first reaction portion in a state in which the first reaction portion is filled with a liquid up to the highest possible liquid level at which the liquid does not overflow, and calcium ions are provided in the liquid regeneration portion of the second reaction portion. Here, the predetermined time may be the time taken to reach a state in which conversion of carbon dioxide is stabilized in the first reaction portion after the start of supply of carbon dioxide. In other words, the predetermined time may be the time taken to reach a state in which variation in amount of produced calcium carbonate is minimized, that is, the variation in amount of produced calcium carbonate per hour is 10% or less, more preferably, 9% or less, 8% or less, 7% or less, 6% or less, 5% or less, 4% or less, 3% or less, 2% or less, or 1% or less.

Also, according to one exemplary embodiment of the present disclosure, in order to disperse the bicarbonate ions obtained by conversion and lower the concentration of bicarbonate ions obtained by conversion around the carbonic anhydrase, the liquid 111 in the first reaction portion 100 may be stirred to prevent stagnation of the liquid 111, particularly the liquid near the liquid surface, and to this end, the first reaction portion 100 may further include a stirring portion 160. Through this, it may be advantageous for preventing or minimizing a reduction in carbon dioxide conversion efficiency of the conversion reaction portion 110. The stirring portion 160 may use any known method of stirring a liquid, and for example, an impeller or a magnetic bar may be used as the stirring portion 160.

The liquid drained through an overflow in the above-described first process is supplied to the second reaction portion 200 to perform the second process, and the second process is an operation in which bicarbonate ions contained in the liquid introduced from the first reaction portion 100 are removed to regenerate the liquid into a liquid from which bicarbonate ions are removed.

The second process is performed in the second reaction portion 200 of the carbon dioxide conversion system 1000. The second reaction portion 200 includes a second chamber 201 having an empty space therein to accommodate a liquid introduced from the first process, a second inlet 220 through which the liquid is supplied into the second chamber 201 from the first reaction portion 100, a second outlet 230 through which a regenerated liquid is discharged to the outside, and a liquid regeneration portion 210 configured to remove bicarbonate ions contained in the introduced liquid. The second reaction portion 200 may further include a product outlet 240 configured to discharge a product obtained by conversion due to the bicarbonate ion removal reaction.

Unlike in FIG. 1, the liquid drained from the first reaction portion 100 in the first process may be supplied to the second reaction portion 200 through a predetermined device, e.g., a pump. However, in this case, there is a difficulty that the flow of liquid in the carbon dioxide conversion system 1000 should be separately controlled at various positions, e.g., between a liquid storage 400 configured to supply a new liquid to the first reaction portion 100 and the first reaction portion 100 and between the first reaction portion 100 and the second reaction portion 200, and there are concerns that the scale of the carbon dioxide conversion system may be increased due to requiring additional energy for pump operation, and an excessive amount of energy may be consumed as the period during which carbon dioxide is continuously converted is prolonged.

Thus, according to one embodiment of the present disclosure, in order to supply the liquid drained from the first reaction portion 100 to the second reaction portion 200 without the addition of separate energy, the liquid may be supplied to the second reaction portion 200 through natural drainage. Also, to this end, the first chamber 101, the first outlet 130, the second chamber 102, and the second inlet 220 may have structural means designed to enable natural drainage using potential energy. As an example of the structural means, the first chamber 101 may be installed at a higher position than the second chamber 102 based on the mean sea level, or the second inlet 220 may be formed at a lower position than the first outlet 130 based on the mean sea level. Meanwhile, even when the second inlet 220 is formed at a lower position than the first outlet 130 based on the mean sea level, the second inlet 220 being formed at a higher point than the highest possible liquid level of the liquid accommodated in the second chamber 201 may be advantageous for smoothly supplying the liquid to the second reaction portion 200 through natural drainage.

Also, as illustrated in FIG. 4, the liquid drained from the first reaction portion 100 may be supplied to the second reaction portion 200 through a conduit connecting the first outlet 130 and the second inlet 220, or as illustrated in FIG. 5, the liquid drained from the first reaction portion 100 may be supplied to a second reaction portion 200′ by falling to a second inlet 220′ formed by an upper portion of the second reaction portion 200′ being entirely or partially open.

Bicarbonate ions are removed from the liquid introduced through the second inlet 220 so that the liquid may be reintroduced into the first reaction portion 100, and the removal of bicarbonate ions may be performed through the liquid regeneration portion 210. The liquid regeneration portion 210 is configured to perform a known method of removing bicarbonate ions from the liquid 111, and for example, the liquid regeneration portion 210 may be a separation membrane capable of separating bicarbonate ions. Alternatively, the liquid regeneration portion 210 may contain calcium ions, specifically, include an aqueous solution containing calcium ions, and may, through calcium ions, ultimately convert bicarbonate ions into calcium carbonate, which is a secondary product, to remove the bicarbonate ions.

Also, the liquid 111 regenerated through the second process may be discharged through the second outlet 230, the secondary product obtained by converting bicarbonate ions into calcium carbonate may be discharged through a calcium carbonate outlet 240 and stored in a product storage 700, and the discharge of calcium carbonate through the calcium carbonate outlet 240 may be controlled through a third valve 840. Here, in consideration of a thickness of a calcium carbonate precipitate 250, the calcium carbonate outlet 240 may be formed at a lower end or lower portion of one side of the second chamber 201 where it is easy to discharge calcium carbonate from the second chamber 201. Also, the second outlet 230 may be formed to be disposed at a point higher than the thickness of the calcium carbonate precipitate 250 so that it is easy to separate and discharge the remaining regenerated liquid excluding the calcium carbonate precipitate 250.

Meanwhile, according to one embodiment of the present disclosure, the second process may be run in a batch circulation manner in which a plurality of second reaction portions each having the liquid regeneration portion containing calcium ions are sequentially added to the second process, and the second reaction portion, which has completed the second process, is added to the second process again. Because the above-described first process is continuously run without pause, carbon dioxide introduced into the first reaction portion 100 is continuously converted into bicarbonate ions, and the bicarbonate ions obtained by conversion are drained together with an overflowing liquid and continuously supplied to the second reaction portion. The bicarbonate ion removal efficiency of the liquid regeneration portion 210 in the second reaction portion 200, the time taken for the removal, the amount of liquid that can be accommodated in the second reaction portion 200, and the like are inevitably limited, and thus with an increase in the scale of the carbon dioxide conversion system 1000, it may become more difficult to continuously run the carbon dioxide conversion system 1000 through a single second reaction portion 200. Accordingly, as illustrated in FIG. 6, the second process may be run in a batch circulation manner in which a plurality of second reaction portions 200A, 200B, 200C, and 200D are added to the second process, and the second reaction portion 200D, which has completed the second process, is added to the second process again. Also, a moving device 900 configured to move the plurality of second reaction portions 200A, 200B, 200C, and 200D so that, after any one second reaction portion 200A finishes receiving a liquid from the first reaction portion, another second reaction portion 200D sequentially receives a liquid from the first reaction portion 100 may be further provided, and for example, the moving device 900 may be a conveyor belt.

Specifically, referring to FIG. 6, in any one second reaction portion 200B which is disconnected from the first reaction portion 100 after receiving a liquid from the first reaction portion 100 for a predetermined amount of time, bicarbonate ion removal may be performed to ultimately convert the remaining bicarbonate ions, unremoved while receiving the liquid, into the calcium carbonate precipitate 250 over a certain period of time. Then, in the second reaction portion 200C in which regeneration of the received liquid is completed by removing bicarbonate ions therefrom, that is, in which the second process is completed, a regenerated liquid discharge process in which the regenerated liquid, excluding the calcium carbonate precipitate 250, in the second reaction portion 200C is separated and discharged and a precipitate discharge process in which the calcium carbonate precipitate 250 is discharged from the second reaction portion 200C from which the liquid has been discharged may be further performed.

Meanwhile, in addition to containing the liquid component originating from the first reaction portion, the regenerated liquid may further contain calcium ions included in the liquid regeneration portion 210 in the second reaction portion 200. Accordingly, when the regenerated liquid containing a large amount of calcium ions is resupplied to the first reaction portion 100, there is a concern that bicarbonate ions obtained by conversion in the first reaction portion 100 may be converted into calcium carbonate in the first reaction portion 100. In particular, calcium carbonate may precipitate after adhering near the liquid surface where the concentration of bicarbonate ions is high, that is, around carbonic anhydrase, for example, a surface of a floating body to which the carbonic anhydrase is fixed, and in this case, because it is not easy for carbonic anhydrase, carbon dioxide, and the liquid to come in contact with each other, there is a concern that carbon dioxide conversion efficiency may be reduced. Accordingly, a selective regenerated liquid processing process may be performed in which, as a result of measuring the concentration of calcium ions in the regenerated liquid, the regenerated liquid is supplied to another second reaction portion, which is in a state in which both the liquid and the precipitate have been discharged, and, as the second reaction portion 200D in which the liquid regeneration portion is formed again, added to the second process again in a case in which the calcium ions are contained in a predetermined concentration or higher, and the regenerated liquid is added to the third process in a case in which the calcium ions are contained in a concentration lower than the predetermined concentration.

A third process in which the liquid regenerated through the above-described second process is directly or indirectly resupplied to the first reaction portion 100 may be performed, the regenerated liquid resupplied to the first reaction portion 100 may constitute the conversion reaction portion 110, and due to the introduced liquid, the liquid near the liquid surface that contains bicarbonate ions obtained by conversion of carbon dioxide may repeat a circulation cycle in which the liquid continues to overflow and be added to the second process 200 from the first reaction portion 100, and in this way, the amount of water and energy required for the carbon dioxide conversion process can be reduced, and carbon dioxide conversion efficiency can be stably maintained high.

Referring to FIG. 1, the liquid regenerated in the second reaction portion 200 may be discharged through the second outlet 230 and directly supplied as a new liquid to the first reaction portion 100 through a pump, e.g., a second pump 320. However, in the carbon dioxide conversion system 1000 illustrated in FIG. 1, because the regenerated liquid cannot be supplied from the second reaction portion 200 to the first reaction portion 100 immediately after the carbon dioxide conversion system 1000 starts to run, the first reaction portion 100 may receive a new liquid through the separate liquid storage 400 for a predetermined amount of time. Also, it should be noted that, because the amount of regenerated liquid recovered through the second reaction portion 200 may not be sufficient in some cases, the first reaction portion 100 may receive a new liquid through the liquid storage 400 even in such cases.

Here, the new liquid may be supplied from the liquid storage 400 to the first reaction portion 100 through a first pump 300, and while the first pump 300 is operated, a first valve 810 may be opened, a second valve 850 may be closed, and the second pump 320 may not be operated. Also, conversely, in a case in which the liquid regenerated in the second reaction portion 200 is supplied as a new liquid, the operation of the first pump 300 may be stopped, the first valve 810 may be closed, the second valve 850 may be opened, and the second pump 320 may be operated. That is, a flow rate of the new liquid supplied to the first reaction portion 100 may be controlled by a single first pump 300 or second pump 320, and when the system is designed so that the liquid supplied from the first reaction portion to the second reaction portion is supplied by natural drainage, both the flow rate of the new liquid supplied to the first reaction portion 100 and the flow rate of the liquid supplied from the first reaction portion 100 to the second reaction portion 200 may be controllable by a single first pump or second pump.

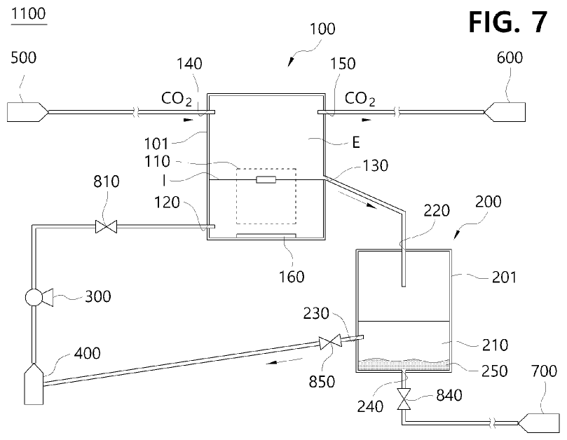

Meanwhile, as illustrated in FIG. 7, the liquid regenerated in the second reaction portion 200 may be supplied to the liquid storage 400 instead of the first reaction portion 100 and may be indirectly resupplied to the first reaction portion 100 via the liquid storage 400. As described above, in the carbon dioxide conversion system 1000 according to FIG. 1, the amount of liquid regenerated in the second reaction portion 200 may be insufficient for a flow rate of a new liquid that should be supplied to the first reaction portion 100, and in this case, there is a difficulty that all of the first pump 300, the second pump 320, the first valve 810, and the second valve 850 should be controlled.

However, in a carbon dioxide conversion system 1100 according to FIG. 7, because it is possible to separately run the process in which the liquid regenerated in the second reaction portion 200 is recovered to the liquid storage 400 and the process in which the new liquid is supplied from the liquid storage 400 to the first reaction portion 100, it may be easier to control the liquid. In particular, there is an advantage that both the flow rate of the new liquid supplied to the first reaction portion 100 and the flow rate of the liquid supplied from the first reaction portion 100 to the second reaction portion 200 can be controlled through the first pump 300. Also, in the case in which the system is configured to have structural means designed so that, as illustrated in FIG. 2, the regenerated liquid is delivered to the liquid storage 400 by natural drainage instead of using a pump that uses energy, there is an advantage that recovery of the regenerated liquid does not require separate energy consumption in addition to opening the second valve 850. Here, the structural means may be implemented by appropriately utilizing a known system design such as forming a height difference between the second chamber 201 and the liquid storage 400 based on the mean sea level.

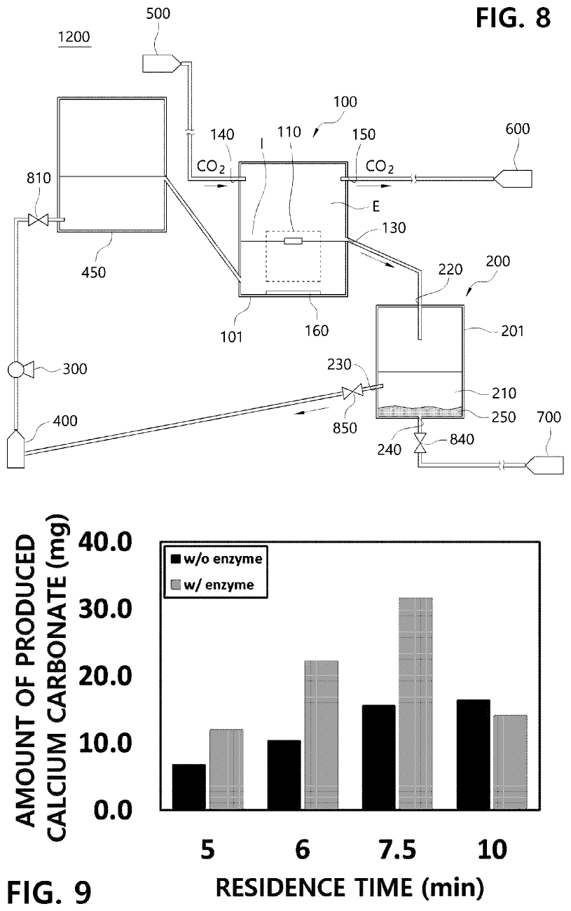

According to one embodiment of the present disclosure, as illustrated in FIG. 8, the new liquid may be supplied to the first reaction portion 100 via an intermediate liquid storage 450 disposed between the liquid storage 400 and the first reaction portion 100. In particular, by including the liquid storage 400, there are advantages that liquids sequentially regenerated in the plurality of second reaction portions can be easily collected and stored, a liquid can be supplied with a desired flow velocity to the intermediate liquid storage 450, and it is convenient when pausing or restarting the entire process. Also, when the new liquid is supplied by natural drainage from the intermediate liquid storage 450 to the first reaction portion 100, as in FIG. 7 described above, both a liquid flow in the first process and a liquid flow in the second process can be controlled through the first pump 300. However, the liquid storage 400 and the intermediate liquid storage 450 may be implemented as a single storage.

### Modes of the Invention

The present disclosure will be described in more detail using the following examples, but the following examples do not limit the scope of the present disclosure and should be construed as being provided to help understanding of the present disclosure.

### Example 1

A first reaction portion was prepared by filling a 1 L-volume first chamber with 300 mL of Tris buffer corresponding to the highest possible liquid level at which the liquid does not overflow, causing a conversion reaction portion, in which carbonic anhydrase fixed by the enzyme adsorption, precipitation, and crosslinking (EAPC) method to a total of 24 mg of nanofibers having an average diameter of 2.0±0.4 μm is fixed to a mesh portion of a 2 cm×6 cm floating body having a mesh formed at an inner side of an edge thereof, to float on the surface of the liquid, disposing a stirring bar as a stirring portion on a lower surface of the first chamber, and forming a carbon dioxide inlet and a carbon dioxide outlet at an upper end of the first chamber so that the carbon dioxide inlet and the carbon dioxide outlet pass through an upper portion of the liquid in the first reaction portion. Then, a second reaction portion having a 50-mL accommodation space and 10 mL of 670 mM calcium chloride for production of calcium carbonate were prepared, and a height difference of 10 cm was formed between a first outlet of the first reaction portion and a second inlet of the second reaction portion to design a carbon dioxide conversion system (A) in which a liquid overflowing from the first reaction portion is naturally drained to the second reaction portion.

### Comparative Example 1

A carbon dioxide conversion system (B) was designed in the same manner as in Example 1 except that carbonic anhydrase was not included in a conversion reaction portion.

### <Experimental Example 1>—Determination of Flow Rate at which New Liquid is Supplied

In the carbon dioxide conversion systems of Example 1 and Comparative Example 1, carbon dioxide gas was supplied at a rate of 150 mL per minute to the first reaction portion, Tris-buffer was supplied as a new liquid at a flow rate of 30 mL per minute, 40 mL per minute, 50 mL per minute, and 60 mL per minute to the first reaction portion, and a stirring bar was rotated at a speed of 100 rpm to run the carbon dioxide conversion systems. Then, the efficiency of the carbon dioxide conversion reaction in the first reaction portion was identified by measuring the amount of calcium carbonate produced in the second reaction portion from a point in time at which carbon dioxide and the new liquid began to be supplied to a point in time at which the amount of produced calcium carbonate per hour was stabilized, which is shown in FIG. 9. Here, in FIG. 9, the residence time refers to a value obtained by dividing 300 mL, which is the largest possible volume of the first reaction portion that does not cause an overflow, by the flow rate (mL/minute) at which the new liquid is supplied. Also, in FIG. 9, “w/enzyme” indicates a result of the carbon dioxide conversion system (A), and “w/o enzyme” indicates a result of the carbon dioxide conversion system (B).

As can be confirmed through FIG. 9, it can be seen that the carbon dioxide conversion efficiency is higher in the carbon dioxide conversion system (A) including carbonic anhydrase, compared to the carbon dioxide conversion system (B) not including carbonic anhydrase.

However, in the case of the carbon dioxide conversion system (A) including carbonic anhydrase, it can be seen that the amount of produced calcium carbonate when the flow rate at which the new liquid is supplied was 30 mL/minute, that is, when the residence time was 10 minutes, was smaller by 50% or more, compared to when the residence time was 7.5 minutes, and it can be seen that the amount of produced calcium carbonate was small even compared to the carbon dioxide conversion system (B) not including carbonic anhydrase, and the carbon dioxide conversion efficiency of the first reaction portion rather decreased despite including carbonic anhydrase. Accordingly, it can be confirmed that the flow rate at which the new liquid is supplied has a great influence on the carbon dioxide conversion efficiency of the first reaction portion.

Therefore, in the case of the carbon dioxide conversion system (A) of Preparation Example 1 including carbonic anhydrase, the flow rate at which the new liquid is supplied while running the carbon dioxide conversion system may be determined as a flow rate exceeding a flow rate that makes the amount of produced calcium carbonate equal to the amount of produced calcium carbonate of the carbon dioxide conversion system (B) not including carbonic anhydrase (any flow rate between 30 mL/minute and 40 mL/minute), and in this way, it is very advantageous for achieving higher carbon dioxide conversion efficiency.

Embodiments of the present disclosure have been described above, but the spirit of the present disclosure is not limited to the embodiments presented herein, and although those of ordinary skill in the art who understand the spirit of the present disclosure may easily propose other embodiments by adding, changing, or omitting components within the scope of the same spirit, such embodiments also belong to the scope of the spirit of the present disclosure.

1. A continuous carbon dioxide conversion process comprising:
a first process in which carbon dioxide introduced into a first reaction portion is converted into bicarbonate ions through a conversion reaction portion including a liquid, which includes water, and carbonic anhydrase positioned in the vicinity of the surface of the liquid, a new liquid is supplied into the first reaction portion, and a liquid exceeding a predetermined liquid level in the first reaction portion due to the supplied new liquid overflows and is supplied to a second reaction portion;
a second process in which the liquid containing the bicarbonate ions, which is introduced into the second reaction portion through the first process, is regenerated into a liquid from which the bicarbonate ions are removed through a liquid regeneration portion in the second reaction portion; and
a third process in which the regenerated liquid recovered through the second process is directly or indirectly resupplied to the first reaction portion.

2. The continuous carbon dioxide conversion process of claim 1, wherein:
the new liquid is supplied from a liquid storage to the first reaction portion; and
in the third process, the regenerated liquid is indirectly resupplied by being supplied to the first reaction portion after being supplied to the liquid storage.

3. The continuous carbon dioxide conversion process of claim 2, wherein:
in order to control both a flow rate of the new liquid supplied from the liquid storage to the first reaction portion and a flow rate of the liquid supplied from the first reaction portion to the second reaction portion through a single first pump, the liquid overflowing from the first reaction portion and being supplied to the second reaction portion is supplied by natural drainage; and
in the third process, the regenerated liquid is supplied to the liquid storage through natural drainage.

4. The continuous carbon dioxide conversion process of claim 2, wherein:
the new liquid is supplied to the first reaction portion via an intermediate liquid storage disposed between the liquid storage and the first reaction portion;
in order to control both a flow rate of the new liquid supplied from the liquid storage to the first reaction portion and a flow rate of the liquid supplied from the first reaction portion to the second reaction portion through a single first pump, the new liquid supplied from the intermediate liquid storage to the first reaction portion and the liquid supplied from the first reaction portion to the second reaction portion are each supplied by natural drainage; and
in the third process, the regenerated liquid is supplied to the liquid storage through natural drainage.

5. The continuous carbon dioxide conversion process of claim 1, wherein the carbon dioxide introduced into the first reaction portion is carbon dioxide contained in a flue gas or carbon dioxide separated and refined from a flue gas.

6. The continuous carbon dioxide conversion process of claim 1, wherein the carbon dioxide introduced into the first reaction portion has a gas flow in which a residual amount unconverted after being introduced from a portion above the surface of the liquid flows out above the surface of the liquid.

7. The continuous carbon dioxide conversion process of claim 1, wherein, in order to position the carbonic anhydrase in the vicinity of the liquid surface without being affected by a change in liquid level and prevent loss of the carbonic anhydrase at the time of discharge of the overflowing liquid, the carbonic anhydrase is provided to be fixed to a floating body, and the floating body is provided to not exit the first reaction portion.

8. The continuous carbon dioxide conversion process of claim 1, wherein the vicinity of the liquid surface is a liquid region from the surface to a point where a depth is 10 cm.

9. The continuous carbon dioxide conversion process of claim 1, wherein, in order to increase a rate at which carbon dioxide is converted into bicarbonate ions in the first reaction portion, the flow rate of the new liquid supplied into the first reaction portion is determined as a flow rate that exceeds a flow rate at the time the value of R according to Equation 1 below satisfies 1:
(Amount of calcium carbonate produced in carbon dioxide conversion system A (g))/(Amount of calcium carbonate produced in carbon dioxide conversion system B (g))=R [Equation 1]
wherein the carbon dioxide conversion system (A) is a carbon dioxide conversion system in which the conversion reaction portion including the carbonic anhydrase is disposed in the first reaction portion, the carbon dioxide conversion system (B) is a carbon dioxide conversion system identical to the carbon dioxide conversion system (A) except for the absence of the carbonic anhydrase in the first reaction portion, and the amount of calcium carbonate produced in the two carbon dioxide conversion systems refers to the amount of calcium carbonate produced in the second reaction portion a predetermined time after the start of supply of carbon dioxide and the new liquid with a predetermined flow rate to the first reaction portion in a state in which the first reaction portion is filled with a liquid up to the highest possible liquid level at which the liquid does not overflow, and calcium ions are provided in the liquid regeneration portion of the second reaction portion.

10. The continuous carbon dioxide conversion process of claim 1, wherein, in order to disperse the bicarbonate ions obtained by conversion in the first process, the liquid in the first reaction portion is stirred in the first process.

11. The continuous carbon dioxide conversion process of claim 1, wherein the liquid regeneration portion includes calcium ions, and by the bicarbonate ions introduced into the liquid regeneration portion being ultimately converted into calcium carbonate through the calcium ions, the bicarbonate ions are removed.

12. The continuous carbon dioxide conversion process of claim 1, wherein the second process is run in a batch circulation manner in which a plurality of second reaction portions each having the liquid regeneration portion containing calcium ions are sequentially added to the second process, and the second reaction portion that has completed the second process is added to the second process again.

13. The continuous carbon dioxide conversion process of claim 12, wherein the second reaction portion that has completed the second process further performs:
a regenerated liquid discharge process in which the regenerated liquid, excluding a calcium carbonate precipitate, in the second reaction portion is separated and discharged;
a precipitate discharge process in which the calcium carbonate precipitate is discharged from the second reaction portion from which the liquid has been discharged; and
a selective regenerated liquid processing process in which, as a result of measuring a concentration of calcium ions in the regenerated liquid, the regenerated liquid is supplied to another second reaction portion, which is in a state in which both the liquid and the precipitate have been discharged, and then added to the second process again in a case in which the calcium ions are contained at a predetermined concentration or higher, or the regenerated liquid is added to the third process in a case in which the calcium ions are contained at a concentration lower than the predetermined concentration.

14. A continuous carbon dioxide conversion system comprising:
a first reaction portion including a first chamber having an empty space therein, a conversion reaction portion including a liquid, which includes water, with which the empty space is filled up to a predetermined liquid level, and carbonic anhydrase positioned in the vicinity of the surface of the liquid, a first inlet disposed at one side of the first chamber to receive a new liquid, and a first outlet provided to allow a liquid exceeding the predetermined liquid level to overflow and be drained;
a second reaction portion including a second chamber having an empty space therein, a second inlet through which the liquid drained after overflowing from the first reaction portion is introduced, a liquid regeneration portion configured to remove bicarbonate ions contained in the introduced liquid and regenerate the liquid, and a second outlet configured to discharge the regenerated liquid; and
a pump configured to directly or indirectly supply the regenerated liquid, discharged through the second outlet, to the first reaction portion.

15. The continuous carbon dioxide conversion system of claim 14, wherein the first reaction portion further includes a carbon dioxide inlet and a carbon dioxide outlet provided in the first chamber so that carbon dioxide has a gas flow in which a residual amount unconverted after being introduced from a portion above the surface of the liquid flows out above the surface of the liquid.

16. The continuous carbon dioxide conversion system of claim 14, wherein a height from the ground to the first outlet is formed higher than a height from the ground to the second inlet so that the liquid overflowing from the first reaction portion is naturally drained into the second reaction portion.

17. The continuous carbon dioxide conversion system of claim 14, wherein the first reaction portion further includes a stirring portion to disperse bicarbonate ions, obtained by conversion, from the conversion reaction portion into the surrounding liquid.

18. The continuous carbon dioxide conversion system of claim 14, wherein:
in the second reaction portion, the liquid regeneration portion contains calcium ions, and a calcium carbonate outlet configured to discharge a calcium carbonate precipitate, obtained by the bicarbonate ions in the introduced liquid being ultimately converted through a reaction with the calcium ions, is further included; and
the second outlet is disposed at a higher point than a thickness of the calcium carbonate precipitate in order to facilitate separation and discharge of the remaining regenerated liquid excluding the calcium carbonate precipitate.

19. The continuous carbon dioxide conversion system of claim 14, further comprising a liquid storage which is a supply source of the new liquid supplied to the first reaction portion and a first pump disposed on a conduit connecting the liquid storage and the first reaction portion,
wherein, a second pump as a pump for delivering the regenerated liquid, discharged through the second outlet, to the liquid storage or a structural device designed to deliver the regenerated liquid to the liquid storage through natural drainage is provided.

20. The continuous carbon dioxide conversion system of claim 14, wherein the second reaction portion is provided as a plurality of second reaction portions, and a moving device configured to move the plurality of second reaction portions so that, after any one second reaction portion finishes receiving a liquid from the first reaction portion, another second reaction portion sequentially receives a liquid from the first reaction portion is further provided.
